(19) United States
(12) Patent Application Publication
(10) Pub. No.: US 2017/0231790 A1
(43) Pub. Date: Aug. 17, 2017

(54) BIORESORBABLE SCAFFOLD DELIVERY SYSTEM WITH IMPROVED DISTAL INTEGRITY

(71) Applicant: Abbott Cardiovascular Systems Inc., Santa Clara, CA (US)

(72) Inventors: Chad J. Abunassar, San Francisco, CA (US); Stephen D. Pacetti, San Jose, CA (US)

(21) Appl. No.: 15/045,849

(22) Filed: Feb. 17, 2016

(51) Int. Cl. A61F 2/915; A61F 2/844; A61F 2/958

(52) U.S. Cl. CPC A61F 2/915; A61F 2/958; A61F 2/844; A61F 2002/91541; A61F 2002/91558; A61F 2002/91583; A61F 2002/91575

(57) ABSTRACT

Delivery systems are disclosed for bioresorbable scaffolds that decrease in length when expanded to a deployment diameter that allow accurate positioning of the scaffold at a lesion. The scaffolds are mounted on a catheter that includes marker bands that are positioned interior to the proximal and distal edges of the crimped scaffold to anticipate the shortening of the scaffold upon deployment. Delivery systems are further disclosed for bioresorbable scaffolds that increase in length when expanded to a deployment diameter that allow accurate positioning of the scaffold at a lesion. The scaffolds are mounted on a catheter that includes marker bands that are positioned exterior to the proximal and distal edges of the crimped scaffold to anticipate the lengthening of the scaffold upon deployment.

### BACKGROUND OF THE INVENTION

Field of the Invention

This invention relates to polymeric medical devices, in particular, systems for delivery or deployment of bioresorbable scaffolds.

Description of the State of the Art

This invention relates to radially expandable endoprostheses that are adapted to be implanted in a bodily lumen. An “endoprosthesis” corresponds to an artificial device that is placed inside the body. A “lumen” refers to a cavity of a tubular organ such as a blood vessel. A stent is an example of such an endoprosthesis. Stents are generally cylindrically shaped devices that function to hold open and sometimes expand a segment of a blood vessel or other anatomical lumen such as urinary tracts and bile ducts. Stents are often used in the treatment of atherosclerotic stenosis in blood vessels. “Stenosis” refers to a narrowing or constriction of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

Stents are typically composed of a scaffold or scaffolding that includes a pattern or network of interconnecting structural elements or struts, formed from wires, tubes, or sheets of material rolled into a cylindrical shape. This scaffolding gets its name because it physically holds open and, if desired, expands the wall of the passageway. Typically, stents are capable of being compressed or crimped onto a catheter so that they can be delivered to and deployed at a treatment site.

Delivery includes inserting the stent through small lumens using a catheter and transporting it to the treatment site. Deployment includes expanding the stent to a larger diameter once it is at the desired location. Mechanical intervention with stents has reduced the rate of restenosis as compared to balloon angioplasty. Yet, restenosis remains a significant problem. When restenosis does occur in the stented segment, its treatment can be challenging, as clinical options are more limited than for those lesions that were treated solely with a balloon.

To assist in accurate placement of the catheter and stent at the lesion site it is useful to visually monitor the catheter as it advances through a vessel. Fluoroscopes or other similar X-ray emitting devices are used to view the catheter within the body as it is advanced. However, in order for the catheter to be visible when exposed to X-rays, the catheter or a portion of the catheter must be radiopaque to X-rays. In catheter designs, radiopaque marker bands or catheter tips are often attached to the catheter for this purpose. For example, radiopaque marker bands have been placed on the inner shaft of the catheter on either side of the stent mounted to the balloon to mark the ends of the stent during delivery. One problem with such bands it that they locally stiffen the catheter shaft and thereby impart an undesirable discontinuity thereto as the metal radiopaque bands are relatively inflexible compared to a polymer balloon catheter shaft.

Stents are generally made to withstand the structural loads, namely radial compressive forces, imposed on the scaffold as it supports the walls of a vessel. Therefore, a stent must possess adequate radial strength if its function is to support a vessel at an increased diameter. Radial strength, which is the ability of a stent to resist radial compressive forces, relates to a stent's radial yield strength and radial stiffness around a circumferential direction of the stent. A stent's “radial yield strength” or “radial strength” (for purposes of this application) may be understood as the compressive loading or pressure, which if exceeded, creates a yield stress condition resulting in the stent diameter not returning to its unloaded diameter, i.e., there is irrecoverable deformation of the stent.

Some treatments with stents require its presence for only a limited period of time. Once treatment is complete, which may include structural tissue support and/or drug delivery, it may be desirable for the stent to be removed or disappear from the treatment location. One way of having a stent disappear may be by fabricating a stent in whole or in part from materials that erode or disintegrate through exposure to conditions within the body. Stents fabricated from biodegradable, bioresorbable, bioabsorbable, and/or bioerodable materials such as bioresorbable polymers can be designed to completely erode only after the clinical need for them has ended. Achieving adequate radial strength is a challenge for such bioresorbable stents since polymers are weaker than the metals used to construct conventional stents.

### SUMMARY

Embodiments of the present invention include a delivery system for a bioresorbable scaffold comprising: a catheter; a balloon disposed over the catheter; a bioresorbable scaffold in a crimped configuration over the catheter comprising a plurality of connected undulating cylindrical rings including crests, wherein two or more of the crests on adjacent rings are connected from a peak of one to a peak of the other and the connected crests point toward each other, wherein when the scaffold is expanded a length of the scaffold decreases; and a proximal marker band and distal marker band disposed over the catheter, wherein the proximal marker band and the distal marker band are interior to a proximal scaffold edge and distal scaffold edge, wherein when the scaffold is expanded to a selected deployment diameter, the proximal marker band is at or overlaps the proximal scaffold edge and the distal marker band is at or overlaps the distal scaffold edge.

Embodiments of the present invention include a delivery system for a bioresorbable scaffold comprising: a catheter; a balloon disposed over the catheter; a bioresorbable scaffold in a crimped configuration over the catheter comprising a plurality of connected undulating cylindrical rings including crests, wherein two or more of the crests on adjacent rings are connected from a peak of one to a peak of the other and the connected crests point toward each other, wherein when the scaffold is expanded a length of the scaffold decreases; a first pair of a proximal marker band and a distal marker band disposed over the catheter, wherein the first pair are interior to a proximal scaffold edge and distal scaffold edge and; a second pair of a proximal marker band and a distal marker band disposed over the catheter, wherein the second pair are positioned between the first pair, wherein when the scaffold is expanded to a nominal balloon diameter, the proximal marker band of the first pair is at or overlapping a proximal scaffold edge and the distal marker band of the first pair is at or overlapping a distal scaffold edge and, wherein when the scaffold is expanded to a post-dilated deployment diameter greater than the nominal deployment diameter, the proximal marker band of the second pair is at or overlapping the proximal edge of the scaffold and the distal marker band is at or overlapping the distal edge of the scaffold.

Embodiments of the present invention include a delivery system for a bioresorbable scaffold comprising: a catheter; a balloon disposed over the catheter; a bioresorbable scaffold composed of a bioresorbable polymer, the scaffold being in a crimped configuration over the catheter and comprising a plurality of undulating cylindrical rings connected by links, wherein a length of the scaffold increases when radially expanded; a proximal marker band disposed over the catheter beyond a proximal scaffold edge; and a distal marker band disposed over the catheter beyond a distal scaffold edge, wherein when the scaffold is expanded to a selected deployment diameter, the proximal marker band is at or overlaps the proximal scaffold edge and the distal marker band is at or overlaps the distal scaffold edge.

Embodiments of the present invention include a method for delivering a bioresorbable scaffold comprising: advancing a delivery system through a vasculature of a patient to a lesion site in a blood vessel, wherein the delivery system comprises a catheter; a balloon disposed over the catheter, a bioresorbable scaffold in a crimped configuration over the catheter, and a pair of marker bands disposed over the catheter interior to a proximal scaffold edge and a distal scaffold edge; monitoring the position of the delivery system with x-ray imaging of the marker bands; positioning the delivery system at the lesion site based on the image of the marker bands; and expanding the scaffold by inflating the balloon to a selected deployment diameter, wherein a length of the scaffold decreases as the scaffold expands and when the scaffold is at the selected deployment diameter, the proximal marker band is at or overlaps the proximal scaffold edge and the distal marker band is at or overlaps the distal scaffold edge.

Embodiments of the present invention include a method for delivering a bioresorbable scaffold comprising: advancing a delivery system through a vasculature of a patient to a lesion site in a blood vessel, wherein the delivery system comprises a catheter; a balloon disposed over the catheter, a bioresorbable scaffold in a crimped configuration over the catheter, and a pair of marker bands disposed over the catheter exterior to a proximal scaffold edge and a distal scaffold edge; monitoring the position of the delivery system with x-ray imaging of the marker bands; positioning the delivery system at the lesion site based on the image of the marker bands; and expanding the scaffold by inflating the balloon to a selected deployment diameter, wherein a length of the scaffold increases as the scaffold expands and when the scaffold is at the selected deployment diameter, the proximal marker band is at or overlaps the proximal scaffold edge and the distal marker band is at or overlaps the distal scaffold edge.

### BRIEF DESCRIPTION OF THE DRAWINGS

FIG. 1 depicts ring structures of a scaffold design that exhibits no or minimal shortening upon expansion.

FIG. 2 depicts a photographic image of stents with a design such as that in FIG. 1 in a crimped configuration over a balloon catheter.

FIG. 3 depicts a close-up view of a leading edge of the bioresorbable scaffold crimped over a balloon catheter illustrating kinking adjacent to a stiff marker band when bending loads are applied.

FIG. 4 depicts a section of a peak-to-peak scaffold design that exhibits shortening upon expansion.

FIG. 5 depicts a section of an offset peak-to-peak scaffold design that exhibits shortening upon expansion.

FIG. 6 depicts a scaffold that shortens upon expansion crimped over a scaffold with markers placed exterior to scaffold edges and the scaffold upon expansion.

FIG. 7 depicts the scaffold of FIG. 6 with marker bands placed interior to scaffold edges crimped over a catheter and the scaffold upon expansion.

FIG. 8A depicts a scaffold crimped over a catheter with marker bands placed on or exterior to scaffold edges and a schematic of the corresponding bending stiffness vs. length.

FIG. 8B depicts a scaffold crimped over a scaffold with marker bands placed interior to scaffold edges and a schematic of the corresponding bending stiffness vs. length.

FIG. 9 illustrates a delivery system having two pairs of marker bands over a catheter that anticipates the shortening of a scaffold when expanded to a nominal deployment diameter and a post-dilated deployment diameter.

FIG. 10 depicts a portion of an exemplary scaffold pattern which lengthens when expanded in a crimped configuration and an expanded configuration.

FIG. 11 depicts expansion of a lengthening scaffold with marker bands exterior to the scaffold edges in a crimped configuration and marker bands at the edges at nominal expansion diameter.

### DETAILED DESCRIPTION

The present invention relates to a bioresorbable scaffold delivery system with improved distal integrity via advantageous scaffold length changing behavior or catheter features. The embodiments are generally applicable to balloon expandable stents or scaffolds composed of a network of struts that change length when radially compressed or crimped and when radially expanded.

A radially expandable scaffold can have virtually any structural pattern that is compatible with a bodily lumen in which it is implanted. A structural pattern for a scaffold may include a pattern or network of with a structure of undulating circumferential rings that are connected. The rings may be directly connected or connected by longitudinally extending linking struts. The undulating rings include crests at which the rings deform or bend to allow the radial compression and expansion. The scaffold plastically deforms at the crests when radially compressed to a crimped or reduced configuration and when radially expanded from a crimped configuration to a deployed configuration.

In general, struts are designed to contact the lumen walls of a vessel and to maintain vascular patency.

The outer diameter of a fabricated scaffold (prior to crimping and deployment) may be between 0.2 to 5.0 mm. For coronary applications, a fabricated scaffold diameter is 2.0 to 5 mm. The length of the scaffold may be 6 to 40 mm or more depending on the application.

A scaffold may be fabricated from a thin-walled tube formed by extrusion, injection molding, coating, or dipping. A scaffold pattern may be formed in the tube with a technique such as laser cutting. A fabricated diameter may correspond to the laser cut diameter of the scaffold. The fabricated diameter may be the same as a selected deployment diameter such as the nominal diameter or it may be 1 to 1.5, 1.1 to 1.3, or 1.3 to 1.5 times a selected deployment diameter. The fabricated diameter may be less than a selected deployment diameter, for example, 0.7 to 0.8, 0.8 to 0.9, or 0.9 to 0.99 times a selected deployment diameter. A crest opening angle, as defined below, of a scaffold pattern at the fabricated diameter or selected deployment diameter may be 80° to 140°, 80° to 100°, 90° to 100°, 100° to 120°, 120° to 130°, or 130° to 140°.

The scaffold in the present invention is composed either partially or completely of a bioresorbable polymer. In general, polymers can be biostable, bioabsorbable, biodegradable, bioresorbable, or bioerodable. Biostable refers to polymers that are not biodegradable. The terms biodegradable, bioabsorbable, bioresorbable, and bioerodable, as well as degraded, eroded, resorbed, and absorbed, are often used interchangeably and refer to polymers that are capable of being completely eroded or absorbed when exposed to bodily fluids such as blood and can be gradually resorbed, absorbed, and/or eliminated by the body. A polymer coating on the surface of a stent body or scaffold may also include a biodegradable polymer which may be a carrier for an active agent or drug.

Thin struts are desirable when designing a scaffold to reduce the radial profile of a deployed scaffold and minimize biological impacts such as neointimal thickness or blood flow disruption. A radial thickness or thickness of the stent body or scaffold may be 80 to 100 microns, 90 to 110 microns, 100 to 120 microns, 120 to 140 microns, 140 to 160 microns, or greater than 160 microns. The ratio of strut width to strut thickness (tube wall thickness) may be 0.7 to 1, 1 to 1.2, 1.2 to 1.5, 1.5 to 1.8, 1.8 to 2, or 2 to 2.5. The radial strength of a scaffold depends on the mechanical properties of the scaffold material (modulus, strength, etc.), pattern design (e.g., number of rings per unit length), and the strut width and thickness.

The polymer of the scaffold may include poly(L-lactide) (PLLA), poly(DL-lactide) (PDLLA), polyglycolide (PGA), poly(D,L-lactide-co-glycolide) (PLGA), poly(L-lactide-co-glycolide), polycaprolactone (PCL), poly(D,L-lactide-co-caprolactone), or poly(L-lactide-co-caprolactone). The polymer may further include blends with or copolymers of poly(L-lactide) with polyglycolide, poly(D,L-lactide-co-glycolide), poly(L-lactide-co-glycolide), polycaprolactone, poly(D,L-lactide-co-caprolactone), or poly(L-lactide-co-caprolactone). Such blends and copolymers may include 80 to 95 wt % or 95 to 99 wt % of PLLA. PLLA and such blends and copolymers with PLLA may be referred to as PLLA-based polymers. The scaffold may further include blends with or copolymers of polydioxanone, polyethylene oxide, polyethylene glycol, poly(butylene succinate), poly(trimethylene carbonate), poly(butylene succinate), or any combination thereof.

The “nominal diameter” or “nominal deployment diameter” may refer to the labeled inflation diameter of a balloon, e.g., a balloon labeled as “3.0 mm” has a nominal diameter or nominal inflation diameter of 3.0 mm which is the outer diameter of the balloon and corresponds to the inner diameter of a scaffold mounted over the balloon. The nominal diameter may be 2.25 mm, 2.5 mm, 2.75 mm, 3 mm, 3.25, mm, 3.5 mm, 3.75 mm, 4 mm or 4.5 mm. A “post-dilated diameter” or “post-dilated deployment diameter” may refer to a diameter beyond the nominal balloon diameter. The nominal to post dilation ratios for a balloon may range from 1.05 to 1.30 (i.e., a post-dilated diameter may be 5% to 30% greater than a nominal inflated balloon diameter). The scaffold diameter, after attaining an inflated diameter by balloon pressure, will to some degree decrease in diameter due to recoil effects related primarily to, any or all of, the manner in which the scaffold was fabricated and processed, the scaffold material and the scaffold design.

PLLA-based polymers are fundamentally weaker than the metals used for constructing metallic stents such as bare metal stents and drug eluting stents. Therefore, two problems become apparent when designing thin strutted PLLA scaffolds to treat coronary blockages. The first problem is that PLLA-based scaffolds have reduced radial strength compared to metal stents and require more material volume (strut width and thickness) to achieve the same radial strength as a metallic stent. This makes the design of a thin strut PLLA-based scaffold with adequate strength especially difficult when also attempting to minimize the profile of the device.

The second problem is that thin strut PLLA-based scaffolds are more likely to experience severe flaring damage during tracking across anatomic obstacles such as sharp calcified plaques. Struts can potentially catch on a calcified plaque when localized kinking of the delivery system occurs during tracking across a tortuous lesion. Both problems are further exacerbated by the use of ultra-thin polymer struts (e.g., less than 120 or 100 microns).

With respect to the first problem, current designs that experience little or no length change upon deployment (e.g., less than 10%) can be placed accurately at a lesion site since the location of the marker bands with respect to the scaffold does not change or changes very little when the scaffold is expanded from the crimped state to the deployed state. However, such designs may have reduced or insufficient radial strength due to limitations on the number of rings per unit length.

Accurate stent placement is critical for minimizing post-procedural restenosis. In other words, ideally a deployed stent must cover the entire length of a lesion without extending substantially into the healthy vessel segments on either side of the target lesion. To reduce the chance of a lesion miss (geographic miss), many manufacturers have adopted peak-to-valley connections in their stent and bioresorbable scaffold designs. A “peak” refers to the outer or convex side or portion of a crest. A “valley” refers to the inner or concave side or portion of a crest. Such designs result in either no length change or little length change when deployed from the crimped state. The peak-to-valley designs include a ring pattern of two or more links connecting adjacent rings from the valley of a crest on one ring to the peak of a crest on the adjacent ring.

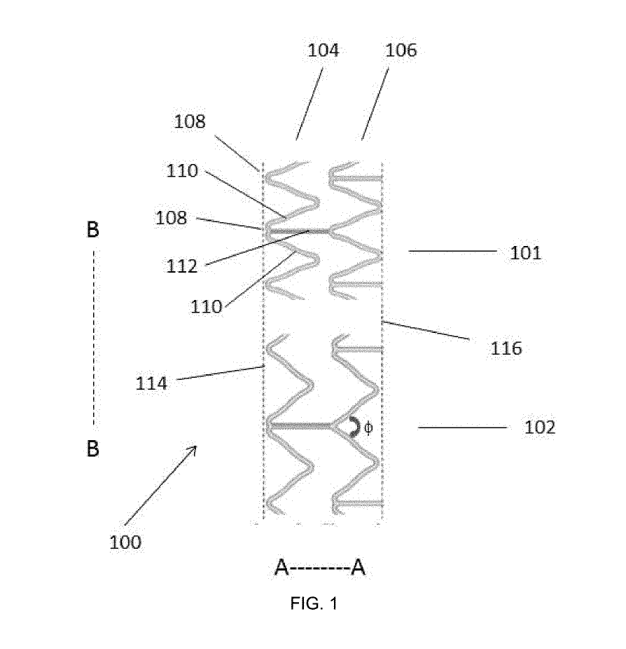

FIG. 1 depicts two rings (104, 106) of a peak-to-valley scaffold design 100 in a flattened view in an initial configuration 101 and an expanded configuration 102. The longitudinal axis of the scaffold is represented by A-A and the circumferential direction by B-B. Dashed lines 114, 116 on either side of the two rings illustrate that the design exhibits no or minimal shortening upon expansion. Rings 104 and 106 include an undulating series of struts 110 that meet at crests 108. Rings 104 and 106 are connected by linking struts 112 that connect a valley of a crest on ring 104 with a peak of a crest on ring 106. The rings are in phase which means the crests of the rings are longitudinally aligned and point in the same direction.

This design configuration maintains scaffold length between the crimped state and the expanded state, thereby minimizing scaffold shortening during deployment. Since this behavior is well known, the peak-to-valley design has become a choice for several bioresorbable scaffolds proposed for commercial use. While accurate to place, these bioresorbable scaffolds typically have reduced radial strength when compared to similar metallic drug eluting stent (DES) designs, even though the PLLA-based scaffolds have a strut thickness of 150 microns or more versus 81 microns for metallic stents.

With respect to the second problem discussed above, crimped thin strut scaffold systems are prone to kinking and catching in synthetic anatomical model (SAM) testing wherein a scaffold delivery system can be tracked through curved synthetic vasculature with hard calcium-representing lesions designed within the vessel. This problem is associated with local hinge points within the stent delivery system in the region near the marker bands. Stent manufacturers place marker bands on the stent delivery system at both ends of the scaffold/stent or at a known distance outward from or beyond the stent edges. These marker bands allow for stents to be accurately placed at a lesion by providing visibility of the delivery system on either side of the crimped stent during tracking to a target lesion under fluoroscopy. In DES systems, some manufacturers place the stent edges on the marker bands while others place the stent edges interior to the marker bands. In either of these configurations, physicians use the marker bands to judge where the deployed stent will be placed and rely on the fact that only minimal stent shortening will occur.

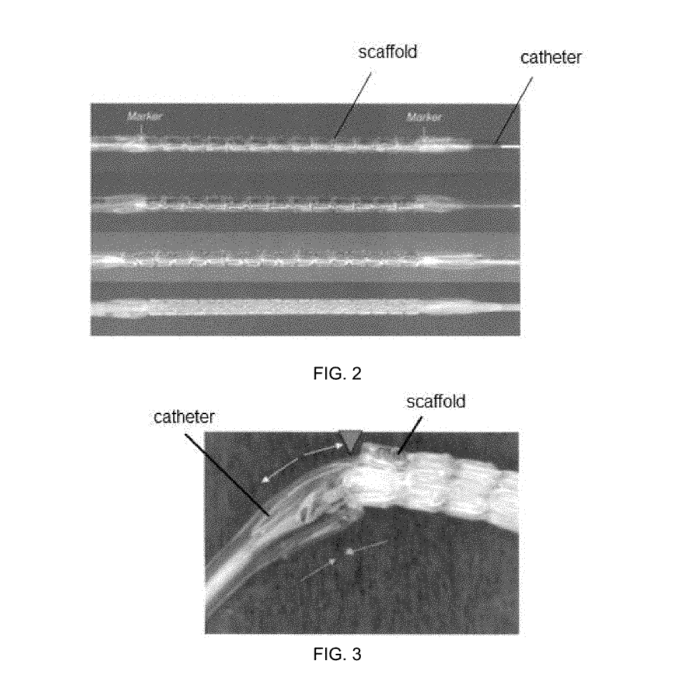

FIG. 2 depicts balloon marker positions relative to stent position on exemplary DES systems. Accurate stent and marker alignment allows for optimal stent positioning which minimizes angiographic miss. However, it has been observed that with thin-strut bioresorbable scaffolds with such designs, positioning the scaffold on or near the marker band results in a potential kinking behavior that exposes the leading edge of a tracking scaffold to calcification. If the scaffold strut catches on a piece of calcium, the struts can flip back or even fracture.

FIG. 3 depicts a bioresorbable scaffold delivery system kinking when bending (scaffold edge placed on marker). The system kinks just adjacent to the stiff marker band when bending loads are applied, where outward pointing arrows depict the tensile side of a bend and inward pointing arrows depict the compressive side of a bend. The triangle represents a calcium spicule that can catch on the leading edge of the exposed strut.

The present invention aims to increase radial strength beyond that of a traditional peak-to-valley design with delivery systems that include scaffold designs that include more rings per unit length of scaffold. In certain embodiments, an intentionally shortening scaffold design is used with the delivery system. Marker bands are intentionally placed interior to the scaffold length at locations that anticipate shortening during deployment. This scaffold design will therefore shorten during deployment and includes more rings per unit length in its deployed state to enhance radial strength. Further, the design will also be less likely to catch on calcified plaques since the distal scaffold portion (i.e., leading edge most likely to catch on calcified plaques) overhangs the high-stiffness marker band region(s) of the delivery system. As shown below, this design will reduce the propensity for kinking near the distal leading edge of the scaffold, thereby reducing the propensity for strut catching, strut flaring, or strut fracture.

This shortening behavior of designs that shorten upon deployment is intrinsic to the kinematics of a peak-to-peak scaffold design, however, this behavior can be somewhat counteracted by any combination of: (1) incorporating a ‘tacky’ balloon surface or material, (2) relying on axial balloon growth during deployment, and/or (3) limiting the expansion range for a particular design such that the shortening kinematics are not severe.

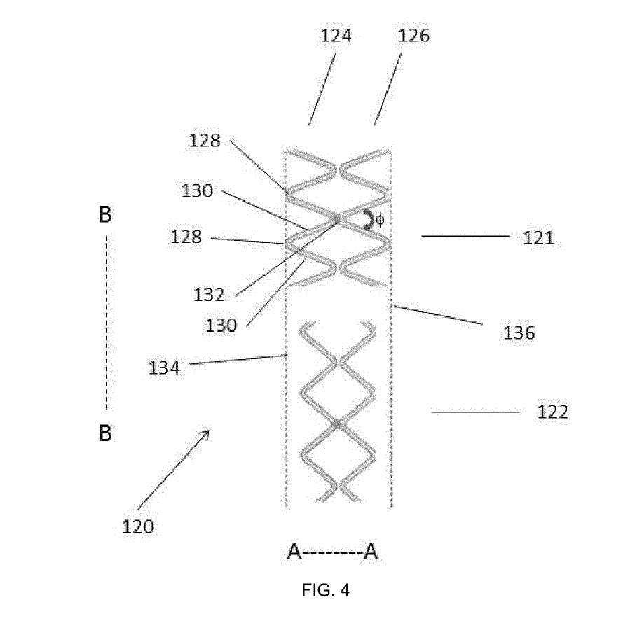

A peak-to-peak design is an example of a pattern that naturally shortens upon deployment. In peak-to-peak scaffold patterns, two or more crests on adjacent rings are connected from the peak of one to the peak of the other and the connected crests point toward each other. The crests are axially aligned or slightly out of phase, which offers dense packing of stent rings. The crests may be directly connected at the crests or may be connected by links. The dense packing in turn allows for tailoring a stent pattern with high radial strength and high radial stiffness. Natural shortening behavior of a peak-to-peak scaffold design is shown below in FIG. 4, which depicts two rings (124, 126) of a peak-to-peak scaffold design 120 in a flattened view in an initial configuration 121 and an expanded configuration 122. The longitudinal axis of the scaffold is represented by A-A and the circumferential direction by B-B. Dashed lines 134, 136 on either side of the two rings illustrate that the design exhibits shortening upon expansion. Rings 124 and 126 include an undulating series of struts 130 that meet at crests 128. Rings 124 and 126 are connected at 132, a peak of a crest on ring 124 with a peak of a crest on ring 126.

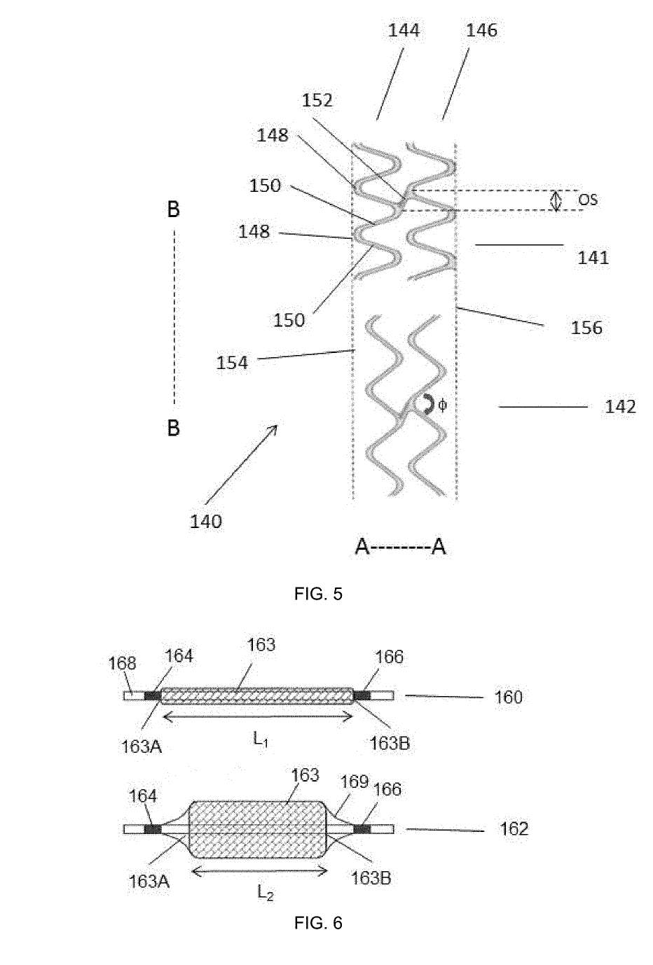

An offset peak-to-peak design is another scaffold design that shortens upon deployment. In an offset peak-to-peak design, the peaks of crests on adjacent rings are connected, point toward each other, but the peaks of the crests are not longitudinally aligned and are offset circumferentially. An offset peak-to-peak pattern excessively shortens under modest (clinically relevant) longitudinal compressive loads. The natural shortening behavior of an offset peak-to-peak stent design is shown in FIG. 5 which depicts two rings (144, 146) of an offset peak-to-peak scaffold design 140 in a flattened view in an initial configuration 141 and an expanded configuration 142. The rings have an offset OS. The longitudinal axis of the scaffold is represented by A-A and the circumferential direction by B-B. Dashed lines 154, 156 on either side of the two rings illustrate that the design exhibits shortening upon expansion. Rings 144 and 146 include an undulating series of struts 150 that meet at crests 148. Rings 144 and 146 are connected by diagonal linking struts 152 that connect a peak of a crest on ring 144 with a peak of a crest on ring 146.

Dramatic and problematic shortening has been observed during deployment of a peak-to-peak bioresorbable scaffold. The deployed scaffold maximizes radial strength since many rings are packed over a shorter final deployed length. However, FIG. 6 illustrates that deployment accuracy is severely compromised since the edges of the scaffold move inwards from the marker bands during deployment. FIG. 6 depicts a delivery system in a crimped configuration 160 and deployed configuration 162. In the crimped configuration 160, scaffold 163 has a length L₁ and is crimped over catheter 168 with proximal marker band 164 at proximal scaffold edge 163A and distal marker band 166 at distal scaffold edge 163B. When scaffold 163 is expanded by balloon 169 to, for example, a nominal deployment diameter, as in deployed configuration 162, scaffold 163 length shortens to a length L₂. Proximal scaffold edge 163A moves away from proximal marker band 164 and distal scaffold edge 163B moves away from distal marker band 166. Therefore, the marker bands do not provide an accurate position of the deployed scaffold.

In the present invention, the problematic shortening is anticipated within the design of the scaffold delivery system and is harnessed to maximize radial strength through the various embodiments.

Certain embodiments of the present invention include a delivery system for a bioresorbable scaffold including a catheter, a balloon disposed over the catheter, a scaffold in a crimped configuration over the catheter, and a proximal marker band and distal marker band disposed over the catheter. The scaffold is made of a bioresorbable polymer and includes a plurality of undulating cylindrical rings including crests. At least two crests of adjacent rings are connected. The scaffold has a design that shortens when the scaffold is radially expanded such as a peak-to-peak design or an offset peak-to-peak design.

Both the proximal and distal marker bands are positioned interior to the proximal and distal edges of the crimped scaffold to anticipate the shortening of the scaffold upon deployment. The proximal marker band is at a first position adjacent, but not at a proximal edge of the scaffold and the distal marker band is at a second position adjacent but not at a distal edge of the scaffold. As the scaffold is expanded to a nominal deployment diameter, the scaffold shortens and the distance between the proximal edge of the scaffold and the proximal marker band and the distance between the distal edge of the scaffold and the distal marker band decreases.

In one embodiment, when the scaffold is expanded to the nominal diameter, the proximal marker band is between the first position and a proximal edge of the scaffold and the distal marker band is between the second position and the distal edge of the scaffold. In another embodiment, when the scaffold is expanded to the nominal deployment diameter, the proximal marker band is at or overlaps a proximal edge of the scaffold and the distal marker band is at or overlaps the distal edge of the scaffold.

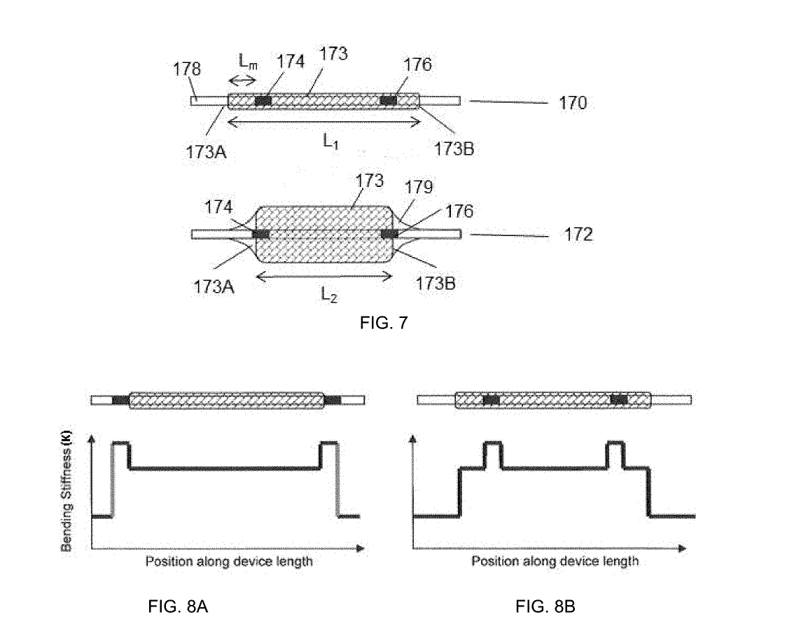

FIG. 7 illustrates a delivery system that anticipates the shortening of a scaffold when expanded so that the marker bands accurately represent the position of the edges of the scaffold when it is deployed. FIG. 7 depicts a delivery system in a crimped configuration 170 and deployed configuration 172. In the crimped configuration 170, scaffold 173 has a length L₁ and is crimped over catheter 178. Proximal marker band 174 and distal marker band 176 are positioned interior to or between proximal scaffold edge 173A and distal scaffold edge 1736. Proximal marker band 174 is at a distance Lₘ distal to proximal scaffold edge 173A and distal marker band 176 is at a distance Lₘ proximal to distal scaffold edge 173B. When scaffold 173 is expanded by balloon 179 to, for example, a nominal deployment diameter, as in deployed configuration 172, scaffold 173 length shortens to a length L₂. Proximal scaffold edge 173A moves toward proximal marker band 174 and distal scaffold edge 1736 moves toward distal marker band 176 resulting in overlap of the scaffold edges and the marker bands. As a result, the marker bands provide an accurate position of the deployed position of the scaffold.

The shortening scaffold designs result in more rings per deployed length, which results in increased radial strength in proportion to the degree of scaffold shortening during deployment. Shortening can be predicted by calculating the cosine of a bar arm opening angle (φ) for a selected deployed diameter in a peak-to-peak design, which can guide the positioning of the marker bands on the delivery system.

In general, a scaffold may have a known amount shortening of Lₛ (L₁-L₂) when expanded from a crimped state to a selected deployment diameter, such as a nominal deployment diameter. Thus, assuming shortening is homogeneous across scaffold length when the scaffold is deployed, when Lₘ is Lₛ/2, the scaffold edges will coincide with the outer edges (proximal edge for proximal marker band and distal edge for distal marker band) of the marker band edges. If the length of a marker band is L_{b}, then a range of Lₘ for overlap of the marker bands with the scaffold edges is: Lₛ/2≦Lₘ≦Lₛ/2+L_{b}.

The percent shortening (% ΔL) may be calculated from the scaffold length in the crimped state (L₁) and the length at the selected deployment diameter (L₂): % ΔL=100%×(L₁−L₂)/L₁. % ΔL may be 1 to 5%, 5 to 10%, 10 to 15%, 10 to 25%, 15 to 20%, 20 to 25%, or 25-30%. Exemplary shortening (ΔL) may be 1 to 2 mm, 2 to 3 mm, 3 to 4 mm, 4 to 5 mm, 5 to 6 mm, 6 to 7 mm, or 7 to 8 mm. Exemplary Lₘ may be 0.5 to 1 mm, 1 to 2 mm, 2 to 3 mm, 3 to 4 mm, or 4 to 5 mm, or 5 to 6 mm for scaffolds with crimped lengths of 18 mm, for example. As coronary scaffolds can have lengths up to 48 mm, exemplary Lₘ may be up to 16 mm.

In addition to increased strength, the delivery system with interior marker bands reduces system kinking that occurs just distal to the scaffold during tracking and delivery. If a scaffold delivery system kinks “just distal” of the scaffold, the leading edge of the distal-most struts are vulnerable to catching on calcium on the outer bend, as shown by the triangle in FIG. 3.

The kinking issue is mitigated with the interior positioning of marker bands as abrupt changes in bending stiffness of the system are reduced substantially as shown in FIGS. 8A-B. FIG. 8A depicts the delivery system bending stiffness along the length (arbitrary scale) for the crimped configuration of FIG. 6 with the marker bands at the edges of the scaffold.

For purposes of this description, the distal portion of the catheter with crimped scaffold can be described as a beam member. The bending stiffness (K) is defined as the resistance of a beam member, such as a catheter, against bending deformation. It is a function of elastic modulus E, the area moment of inertia I of the beam cross-section about the axis of interest, length of the beam, and beam boundary condition. Bending stiffness of a beam can analytically be derived from the equation of beam deflection when it is applied by a force:

K=p/w

where p is the applied force and w is the deflection.

The large and abrupt change in bending stiffness at the scaffold edges can encourage kinking. FIG. 8B depicts the delivery system bending stiffness along the length for the crimped configuration of FIG. 7 with the marker bands at the interior of the scaffold away from the edges. The leading edge of the scaffold is less stiff than in the system of FIG. 6 and is less vulnerable to kinking that results from catching on calcium on the outer bend. The high stiffness marker band still results in a high stiffness region in the delivery system, however, it is away from the distal edge of the scaffold and thus does not contribute to kinking.

In further embodiments, a delivery system with a shortening scaffold includes multiple marker bands placed interior to the edges of the scaffold and balloon such that two or more deployment diameters are anticipated, for example, the scaffold shortening at the nominal and maximum post-dilated deployment diameters.

In such embodiments, a delivery system for a bioresorbable scaffold includes a catheter, a balloon disposed over the catheter, a scaffold in a crimped configuration over the catheter, a first pair of a proximal marker band and distal marker band disposed interior to the edges of the scaffold over the catheter, and a second pair of a proximal marker band and a distal marker band disposed interior to the first pair of marker bands over the catheter. The scaffold is made of a bioresorbable polymer and includes a plurality of undulating cylindrical rings including crests. At least two crests of adjacent rings are connected.

The scaffold has a design that shortens when the scaffold is radially expanded such as a peak-to-peak design or an offset peak-to-peak design.

The first pair of proximal and distal marker bands is positioned interior to the proximal and distal edges of the crimped scaffold to anticipate the shortening of the scaffold upon expansion to a first diameter, such as a nominal deployment diameter. The second pair of proximal and distal marker bands is positioned interior to the first pair of marker bands to anticipate the shortening of the scaffold upon expansion to a second diameter larger than the first diameter, such as a maximum post-dilated deployment diameter.

The proximal marker band of the first pair is at a first position adjacent, but not at a proximal edge of the scaffold and the distal marker band of the first pair is at a second position adjacent but not at a distal edge of the scaffold. As the scaffold is expanded to a nominal deployment diameter, the scaffold shortens and the distance between the proximal edge of the scaffold and the proximal marker band of the first pair and the distance between the distal edge of the scaffold and the distal marker band of the first pair decreases.

When the scaffold is expanded to the nominal diameter, the first pair of the marker bands are positioned as described in the embodiment above with respect to FIG. 7 to allow accurate positioning of the deployed scaffold. When the scaffold is expanded to a post-dilated deployment diameter, such as the maximum allowed post-dilated deployment diameter, the proximal marker band of the second pair is positioned at or overlapping a proximal edge of the scaffold and the distal marker band of the second pair is positioned at or overlapping the distal edge of the scaffold.

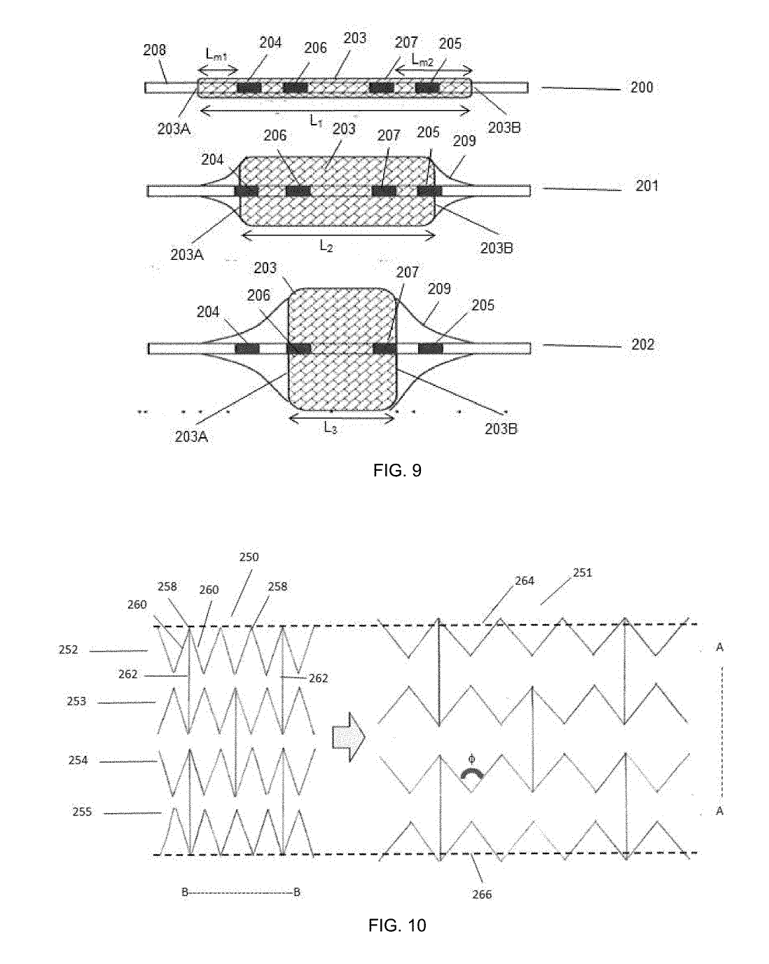

FIG. 9 illustrates a delivery system having two pairs of marker bands over a catheter that anticipates the shortening of a scaffold when expanded to a nominal deployment diameter and a post-dilated deployment diameter so that one of the pairs of marker bands accurately represent the position of the edges of the scaffold when it is deployed to either diameter. FIG. 9 depicts a delivery system in a crimped configuration 200, in a configuration deployed to a nominal diameter 201, and a configuration deployed to a post-dilated diameter 202. In the crimped configuration 200, scaffold 203 has a length L₁ and is crimped over catheter 208. A first pair of marker bands, proximal marker band 204 and distal marker band 205, is positioned interior to proximal scaffold edge 203A and distal scaffold edge 203B. Proximal marker band 204 is at a distance Lₘ₁ distal to proximal scaffold edge 203A and distal marker band 205 is at a distance Lₘ₂ proximal to distal scaffold edge 203B.

A second pair of marker bands, proximal marker band 206 and distal marker band 207, is positioned interior to proximal marker band 204 and distal marker band 205. Proximal marker band 206 is at a distance Lₘ₂ distal to proximal scaffold edge 203A and distal marker band 207 is at a distance Lₘ₂ proximal to distal scaffold edge 203B.

When scaffold 203 is expanded by balloon 209 to, for example, a nominal deployment diameter, as in deployed configuration 201, scaffold 203 length shortens to a length L₂. Proximal scaffold edge 203A moves toward proximal marker band 204 and distal scaffold edge 203B moves toward distal marker band 205 resulting in overlap of the scaffold edges and the marker bands. The second pair of marker bands, proximal marker band 206 and distal marker band 207, are closer to, but still interior to the scaffold edges.

When scaffold 203 is expanded by balloon 209 to a post-dilated deployment diameter that is larger than the nominal diameter, as in deployed configuration 202, scaffold 203 length shortens to a length L₃. Proximal scaffold edge 203A moves toward proximal marker band 206 and distal scaffold edge 203B moves toward distal marker band 207 resulting in overlap of the scaffold edges and these marker bands. As a result, the second pair of marker bands provides an accurate position of the scaffold at the post-dilated deployment diameter.

As above, the scaffold may have a known amount of shortening at each deployment diameter, Lₛ₁ (L₁−L₂) and Ls₂ (L₁−L₃). When Lₘ₁ is Lₛ₁/2, at deployment the scaffold edges will coincide with the outer edges of the first pair of marker band edges and when Lₘ₂ is Lₛ₂/2, at deployment the scaffold edges will coincide with the outer edges of the second pair of marker band edges. Given a length of a marker band, L_{b}, then a range of Lₘ₁ for overlap of the first pair of marker bands with the scaffold edges is Lₛ₁/2≦Lₘ₁≦Lₛ₁/2+L_{b}. and a range of Lₘ₂ for overlap of the second pair of marker bands with the scaffold edges is Lₛ₂/2≦Lₘ₂≦Lₛ₂/2+L_{b}.

The percent shortening for each diameter (% ΔL₂ and % ΔL₃, configuration 201, 202, respectively) may be calculated from the scaffold lengths in the crimped state (L₁) and the lengths at the selected deployment diameters: % ΔL₂=100%×(L₁−L₂)/L₁ and % ΔL₃=100%×(L₁−L₃)/L₁. Values for combinations of (% ΔL₂, % ΔL₃), (ΔL₂, ΔL₃), and (Lₘ₁, Lₘ₂) may correspond to combinations of values provide for % ΔL, ΔL, and Lm.

While two shortened configurations are anticipated and shown in FIG. 9 (nominal and post-dilated deployment), further embodiments include any number of marker bands or pairs of marker bands that correspond to and allow accurate placement of a scaffold at various deployment diameters. The different pairs of markers may have different or alternating levels of radiopacitys to distinguish between markers and thus the corresponding deployment diameters. The varying levels of radiopacitys can be accomplished by selecting different materials for pairs of marker bands such as different alloys of platinum and iridium or by varying marker band thicknesses, i.e., thicker bands will be more radiopaque and appear brighter. Alternatively, the sets of marker bands may be distinguished by varying their size or length L_{b}.

The marker bands in the various embodiments may have the form of a thin-walled cylindrical tube or band. For example, a marker band may have a length of 1 to 3 mm and a wall thickness of 25-75 μm.

In some embodiments, the stiffness of the part of the catheter with the marker band can be reduced through the use of spiral cut marker bands which have greater flexibility or are less stiff than a solid metal tube or band. The spiral-cut marker bands may be placed interior to the scaffold and balloon edges. This allows for smoother bending stiffness transitions along the length of the crimped scaffold, thereby mitigating the possibility of system kinking within the crimped scaffold body. These smooth transitions reduce the possibility of a crimped scaffold from kinking and catching on calcification or other obstacles. The spiral-cut marker bands can be applied to all of the embodiments described herein.

The marker bands of the various embodiments can be made of any radiopaque material that provides visibility under an x-ray fluoroscope. These include metals such as platinum, gold, tantalum, and alloys thereof such as platinum-iridium alloys.

Marker bands can be made of a flexible polymer that has been loaded with radiopaque filler and can also be placed at a position interior to scaffold and at balloon edges. Such marker bands are more flexible than those made of metal. The filler can be particles of platinum, platinum/iridium, tungsten, tantalum, gold, or iodine compounds. The flexible filled polymer marker bands also allow for smoother bending stiffness transitions along the length of the crimped scaffold, thereby mitigating the possibility of system kinking within the crimped scaffold body. The polymeric markers may also be formed to have tapered edges, as opposed to the square edges of a solid metal marker band. The tapered edges give rise to less abrupt changes in crimped scaffold profile and also can lead to less balloon pin-holing during crimping which can occur with solid metal marker bands. In designs where the balloon markers are exposed to the blood, the tapered edges of filled polymeric markers are less likely to cause damage to vascular tissue during delivery.

Embodiments of the present invention further include delivery systems that include scaffolds with patterns that lengthen in a predictable manner when expanded. The degree of lengthening can be predicted by the cosine of the crest opening angle (φ) when the scaffold is deployed to a given diameter. In such delivery systems, marker bands are placed exterior to the scaffold edges in a crimped configuration to anticipate lengthening of the scaffold during deployment. With the marker bands outside of the scaffold during delivery, the higher stiffness, and possible kinking, seen at the marker during delivery in tortuosity will not be aligned with the distal scaffold edge. This will result in less scaffold distal edge flaring. When the scaffold is expanded to a deployed diameter, the scaffold lengthens and the scaffold edges align to the marker bands so that they are at, or overlap, the scaffold edges at the deployed diameter. The deployed diameter can be a nominal or post-dilated diameter.

A scaffold design that lengthens when expanded is referred to as “auxetic” and is said to have auxetic behavior when the scaffold simultaneously lengthens during expansion, and conversely shortens during crimping. Scaffold patterns including connected undulating cylindrical rings can be configured to have auxetic behavior in order to allow the scaffold edges to be positioned at a distance from the relatively stiff catheter marker bands in a first crimped state, while ensuring that the expanded scaffold's edges align with the marker bands in a second expanded state. In the crimped state, the gaps that are present between the scaffold edges and exterior marker bands allow the scaffold delivery system to have reduced bending stiffness transitions and avoid leading edge kinking and subsequent flaring when compared to a system wherein the crimped scaffold edges are positioned on the marker bands (FIG. 8A). Exemplary auxetic scaffold patterns may have a plurality of undulating cylindrical rings that include crests and two or more longitudinal links, that may be longitudinally aligned, connecting adjacent rings from the valley of a crest on one ring to the valley of a crest on the adjacent ring. The connected crests point away from each other.

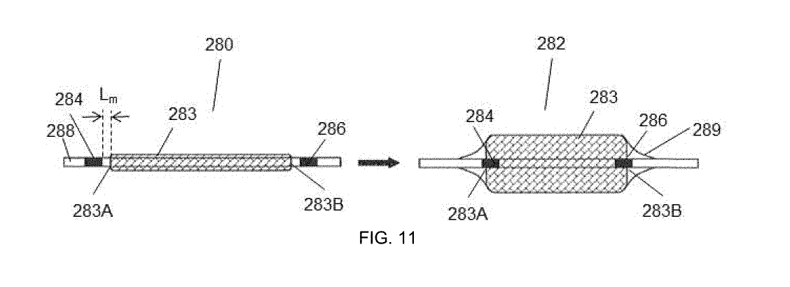

FIG. 10 illustrates an exemplary scaffold pattern which lengthens when expanded. FIG. 10 depicts four rings (252, 253, 254, 255) of an auxetic scaffold design in a flattened view in an initial configuration 250 and an expanded configuration 251. The longitudinal axis of the scaffold is represented by A-A and the circumferential direction by B-B. Dashed lines 264, 266 on either side of the two rings illustrate that the design exhibits lengthening upon expansion. Rings 252 and 253 are connected by linking struts 262 that connect a valley of a crest on ring 252 with a valley of a crest on ring 253. Rings 252, 253, 254, 255 include an undulating series of struts 260 that meet at crests 258.

FIG. 11 illustrates a delivery system that anticipates the lengthening of an auxetic scaffold when expanded so that the marker bands accurately represent the position of the edges of the scaffold when it is deployed. FIG. 11 depicts a delivery system in a crimped configuration 280 and deployed configuration 282. In the crimped configuration 280, scaffold 283 is crimped over catheter 288. Proximal marker band 284 and distal marker band 286 are positioned exterior to proximal scaffold edge 283A and distal scaffold edge 283B, respectively. Proximal marker band 284 is at a distance (Lₘ) proximal to proximal scaffold edge 283A and distal marker band 286 is at a distance (Lₘ) distal to scaffold edge 283B. When scaffold 283 is expanded by balloon 289 to, for example, a nominal deployment diameter, as in deployed configuration 282, scaffold 283 lengthens. Proximal scaffold edge 283A moves toward proximal marker band 284 and distal scaffold edge 283B moves toward distal marker band 286 resulting in overlap of the scaffold edges and the marker bands. As a result, the marker bands provide an accurate position of the deployed position of the scaffold. Embodiments further include additional pairs of marker bands, as in FIG. 9, that anticipate lengthening at additional deployment diameters to allow accurate positioning of the scaffold at these deployment diameters.

In general, a scaffold may have a known lengthening of L₁ when expanded from a crimped state to a selected deployment diameter, such as a nominal deployment diameter. Thus, assuming lengthening is homogeneous across scaffold length, when Lₘ is L₁/2, the scaffold edges will coincide with the inner edges (distal edge for proximal marker band and proximal edge for distal marker band) of the marker band edges. If the length of a marker band is L_{b}, then a range of Lₘ for overlap of the marker bands with the scaffold edges is: Lₛ/2≦Lₘ≦Lₛ/2−L_{b}.

The percent lengthening (% ΔL) may be calculated from the scaffold length in the crimped state (L₁) and the length at the selected deployment diameter (L₂): % ΔL=100%×(L₂−L₁)/L₁. % ΔL may be 1 to 5%, 5 to 10%, 10 to 15%, 10 to 25%, 15 to 20%, 20-25%, or 25-30%. Exemplary lengthening (ΔL) may be 1 to 2 mm, 2 to 3 mm, 3 to 4 mm, 4 to 5 mm, 5 to 6 mm, 6 to 7 mm, or 7 to 8 mm. Exemplary Lₘ may be 0.5 to 1 mm, 1 to 2 mm, 2 to 3 mm, 3 to 4 mm, or 4 to 5 mm for scaffolds with crimped lengths of 18 mm, for example. As coronary scaffolds can have lengths up to 48 mm, exemplary Lₘ may be up to 16 mm.

While this disclosure specifically describes designs for thin-strutted bioresorbable scaffolds, and catheter features, intended to treat coronary artery blockages, the depicted designs can be used for polymeric or metallic implants designed for the treatment of any anatomic lumen.

1. A delivery system for a bioresorbable scaffold comprising:
a catheter;
a balloon disposed over the catheter;
a bioresorbable scaffold in a crimped configuration over the catheter comprising a plurality of connected undulating cylindrical rings including crests, wherein two or more of the crests on adjacent rings are connected from a peak of one to a peak of the other and the connected crests point toward each other, wherein when the scaffold is expanded a length of the scaffold decreases; and
a proximal marker band and distal marker band disposed over the catheter, wherein the proximal marker band and the distal marker band are interior to a proximal scaffold edge and distal scaffold edge,
wherein when the scaffold is expanded to a selected deployment diameter, the proximal marker band is at or overlaps the proximal scaffold edge and the distal marker band is at or overlaps the distal scaffold edge.

2. The delivery system of claim 1, wherein the selected deployment diameter is a nominal deployment diameter.

3. The delivery system of claim 1, wherein the connected crests are connected at the crests.

4. The delivery system of claim 1, wherein the connected crests are connected by links.

5. The delivery system of claim 1, wherein the connected crests are connected by links and are offset circumferentially.

6. The delivery system of claim 1, wherein the proximal marker band, distal marker band, or both are spiral cut marker bands.

7. The delivery system of claim 1, wherein the proximal marker band, distal marker band, or both are composed of a composite of a polymer and a radiopaque material.

8. The delivery system of claim 1, wherein the scaffold comprises a poly(L-lactide)-based polymer.

9. The delivery system of claim 1, wherein the length of the scaffold decreases by 10 to 25% when expanded to the selected deployment diameter.

10. A delivery system for a bioresorbable scaffold comprising:
a catheter;
a balloon disposed over the catheter;
a bioresorbable scaffold in a crimped configuration over the catheter comprising a plurality of connected undulating cylindrical rings including crests, wherein two or more of the crests on adjacent rings are connected from a peak of one to a peak of the other and the connected crests point toward each other, wherein when the scaffold is expanded a length of the scaffold decreases;
a first pair of a proximal marker band and a distal marker band disposed over the catheter, wherein the first pair are interior to a proximal scaffold edge and distal scaffold edge and;
a second pair of a proximal marker band and a distal marker band disposed over the catheter, wherein the second pair are positioned between the first pair,
wherein when the scaffold is expanded to a nominal balloon diameter, the proximal marker band of the first pair is at or overlapping a proximal scaffold edge and the distal marker band of the first pair is at or overlapping a distal scaffold edge and,
wherein when the scaffold is expanded to a post-dilated deployment diameter greater than the nominal deployment diameter, the proximal marker band of the second pair is at or overlapping the proximal edge of the scaffold and the distal marker band is at or overlapping the distal edge of the scaffold.

11. The delivery system of claim 10, wherein the proximal marker band, distal marker band, or both are spiral cut marker bands.

12. The delivery system of claim 10, wherein the proximal marker band of the first pair, distal marker band of the first pair, or both are composed of a composite of a polymer and a radiopaque material.

13. The delivery system of claim 10, wherein the post-dilated deployment diameter is 5% to 30% greater than the nominal deployment diameter.

14. The delivery system of claim 10, wherein the connected crests are connected at the crests.

15. The delivery system of claim 10, wherein the connected crests are connected by links.

16. The delivery system of claim 10, wherein the connected crests are connected by links and are offset circumferentially.

17. The delivery system of claim 10, wherein the scaffold comprises a poly(L-lactide)-based polymer.

18. A delivery system for a bioresorbable scaffold comprising:
a catheter;
a balloon disposed over the catheter;
a bioresorbable scaffold composed of a bioresorbable polymer, the scaffold being in a crimped configuration over the catheter and comprising a plurality of undulating cylindrical rings connected by links, wherein a length of the scaffold increases when radially expanded;
a proximal marker band disposed over the catheter beyond a proximal scaffold edge; and
a distal marker band disposed over the catheter beyond a distal scaffold edge,
wherein when the scaffold is expanded to a selected deployment diameter, the proximal marker band is at or overlaps the proximal scaffold edge and the distal marker band is at or overlaps the distal scaffold edge.

19. The delivery system of claim 18, wherein the proximal marker band, distal marker band, or both are spiral cut marker bands.

20. The delivery system of claim 18, wherein the proximal marker band, distal marker band, or both are composed of a composite of a polymer and a radiopaque material.

21. The delivery system of claim 18, wherein the links connect the crests on adjacent rings from a peak of one to a peak of the other and the connected crests point away from each other.

22. The delivery system of claim 18, wherein the scaffold comprises a poly(L-lactide)-based polymer.

23. The delivery system of claim 18, wherein the length of the scaffold increases by 10 to 25% when expanded to the selected deployment diameter.
